(19) United States
(12) Patent Application Publication
(10) Pub. No.: US 2010/0080841 A1
(43) Pub. Date: Apr. 1, 2010

(54) FIBROUS ARTICLES WITH ONE OR MORE POLYELECTROLYTE LAYERS THEREON AND METHODS FOR MAKING THE SAME

(76) Inventors: Francis E. Porbeni, Woodbury, MN (US); Moses M. David, Woodbury, MN (US); Matthew T. Scholz, St. Paul, MN (US); Prabhakara S. Rao, Maplewood, MN (US)

Correspondence Address: 3M INNOVATIVE PROPERTIES COMPANY, PO BOX 33427, ST. PAUL, MN, 55133-3427, US

(21) Appl. No.: 12/597,059

(22) PCT Filed: Apr. 18, 2008
(86) PCT No.: PCT/US08/60716

§ 371 Date: Oct. 22, 2009

Related U.S. Application Data
(60) Provisional application No. 60/913,384, filed on Apr. 23, 2007.

(51) Int. Cl. A61L 15/00; A61K 33/38; A61F 13/02; A61F 13/00

(52) U.S. Cl. 424/445; 424/618; 427/2.31; 602/48

(57) ABSTRACT

Fibrous articles having fibers coated with one or more polyelectrolyte layers are disclosed. Methods of making and using the fibrous articles are also disclosed.

### TECHNICAL FIELD

This disclosure relates to fibrous articles comprising one or more polyelectrolyte layers. This disclosure also relates to methods of making and using the disclosed fibrous articles.

### SUMMARY

An exemplary aspect of the present disclosure features fibrous articles having enhanced functionality and methods for making fibrous articles having enhanced functionality. An exemplary aspect of the present disclosure also features methods of using fibrous articles having enhanced functionality in a variety of applications.

The surface functionality of the disclosed fibrous article may be customized by providing one or more polyelectrolyte layers on fibers along a first major surface, a second major surface, or both first and second major surfaces of a fibrous substrate. For example, the resulting fibrous article of the present disclosure may have an overall positive surface charge on one or both major outer surfaces, an overall negative surface charge on one or both major outer surfaces, or a combination of positive and negative surface charges on major outer surfaces of the fibrous article. This charge may be pH dependent as discussed herein. In other exemplary embodiments, the fibrous article of the present disclosure may have an overall positive or negative surface charge on one major outer surface, and a neutral surface charge or a surface charge resulting from the material used to form the fibrous substrate.

In one exemplary embodiment, the fibrous article comprises a fibrous substrate (e.g., a nonwoven substrate) comprising fibers along first and second major surfaces of the fibrous substrate; a fiber surface treatment of at least a portion of the fibers along the first major surface, the fiber surface treatment comprising (i) an oxygen plasma treatment, (ii) a first diamond-like glass film coating, or both (i) and (ii); and an anionic polyelectrolyte layer bonded to the fiber surface treatment. The anionic polyelectrolyte layer may be bonded to the fiber surface treatment (e.g., an oxygen plasma treatment on the fiber surface or a first diamond-like glass film coating on the fiber surface) via a bonding layer, such as a bonding layer comprising a silane coupling agent. The fibrous article may further comprise one or more additional polyelectrolyte layers so as to provide a desired functionality to the fibrous article.

In another exemplary embodiment, the fibrous article comprises a fibrous substrate (e.g., a nonwoven substrate) comprising fibers along first and second major surfaces of the fibrous substrate; a diamond-like glass film coating at least a portion of the fibers along the first major surface, the second major surface, or both; a silane coupling layer on the diamond-like glass film; and an anionic polyelectrolyte layer on the silane coupling layer. When the fibrous article comprises a diamond-like glass film coating on the first major surface, the fibrous article may further comprise one or more similar or different coating chemistries on the second major surface of the fibrous article. For example, the fibrous substrate may comprise a first diamond-like glass film coating on the first major surface, and a second diamond-like glass film coating on the second major surface of the fibrous substrate. In other embodiments, the fibrous substrate may comprise a diamond-like glass film coating on the first major surface, and one or more additional layers on the second major surface of the fibrous substrate, such as an adhesive layer.

An exemplary aspect of the present disclosure also features methods of making fibrous articles having enhanced functionality. In one exemplary embodiment, the method of making a fibrous article comprises subjecting a fibrous substrate (e.g., a nonwoven substrate) having first and second major surfaces to a surface treatment process so as to provide a fiber surface treatment over at least a portion of fibers along the first major surface, wherein the fiber surface treatment comprises (i) an oxygen plasma treatment, (ii) a diamond-like glass film coating, or both (i) and (ii); and bonding at least one polyelectrolyte layer to the fiber surface treatment. The method of making a fibrous article may further comprise providing one or more additional layers onto the second major surface of the fibrous substrate, an outer surface of the fiber surface treatment, and/or an outer surface of a polyelectrolyte layer.

In a further exemplary embodiment, the method of making a fibrous article comprises subjecting a fibrous substrate (e.g., a nonwoven substrate) to a plasma deposition process so as to provide a diamond-like-glass film onto fibers of the fibrous substrate; coupling a silane coupling agent to the diamond-like-glass film; and bonding at least one polyelectrolyte layer to the silane coupling agent. In some embodiments, the silane coupling agent comprises amino groups that may be protonated so as to enhance bonding between the silane coupling agent and an anionic polyelectrolyte layer deposited thereon. Further, in some embodiments, the diamond-like-glass film may be treated with an oxygen plasma treatment prior to coupling of the silane coupling agent to the surface treated fibers.

In a further exemplary embodiment, the method of making a polyelectrolyte coated fibrous article comprises subjecting fibers or filaments to a plasma deposition process so as to provide a diamond-like-glass film on the fibers or filaments and subsequently forming the fibers or filaments into a fabric substrate via a nonwoven, knitting or weaving process. A silane coupling agent may be bonded to the diamond-like-glass film and at least one polyelectrolyte layer to the silane coupling agent either before or after forming the diamond-like glass coated fiber into a fabric. In some embodiments, the silane coupling agent comprises amino groups that may be protonated so as to enhance bonding between the silane coupling agent and an anionic polyelectrolyte layer deposited thereon. Further, in some embodiments, the diamond-like-glass film may be treated with an oxygen plasma treatment prior to coupling of the silane coupling agent to the surface treated fibers.

The disclosed methods of making fibrous articles enable the production of functionalized fibrous articles having a desired surface chemistry on outermost surfaces of the fibrous article. Due to the surface properties of the resulting fibrous articles, the fibrous articles have utility in a variety of applications.

An exemplary aspect of the present disclosure also features methods of using the fibrous articles having enhanced functionality in a variety of applications. For example, the fibrous articles are suitable for use in applications including, but not limited to, filtration, microbial detection, wound healing products, drug delivery, bioprocessing (protein purification), permselective materials for protective coatings, food safety, anti-glare and anti-fog materials for medical use, etc.

Other features and advantages of the disclosure will be apparent from the following drawings, detailed description, and claims.

### BRIEF DESCRIPTION OF DRAWING

FIG. 1 depicts a cross-sectional view of an exemplary fiber within a functionalized fibrous article (e.g., a nonwoven fabric) of the present disclosure;

FIG. 2 depicts a cross-sectional view of another exemplary fiber within a functionalized fibrous article of the present disclosure;

FIG. 3 depicts a cross-sectional view of another exemplary fiber within a functionalized fibrous article of the present disclosure;

FIG. 4a depicts a view of an exemplary functionalized nonwoven article of the present disclosure;

FIG. 4b depicts a cross-sectional view of an exemplary fiber along a first major surface of the functionalized nonwoven article of FIG. 4a;

FIG. 4c depicts a cross-sectional view of an exemplary fiber along a second major surface of the functionalized nonwoven article of FIG. 4a;

FIGS. 5a-c depict an exemplary method for forming an exemplary functionalized fibrous article of the present disclosure;

FIG. 6 depicts graphically a sulfur:nitrogen (S/N) atomic ratio for spunbonded web samples of Example 1 versus number of polyelectrolyte layers deposited thereon;

FIG. 7 depicts graphically a detectable amount of atomic silicon concentration on surfaces of spunbonded web samples of Example 1 versus the number of polyelectrolyte layers deposited thereon;

FIG. 8 depicts graphically a sulfur:nitrogen (S/N) atomic ratio for spunbonded web samples of Example 2 versus number of polyelectrolyte layers deposited thereon; and

FIG. 9 depicts graphically a detectable amount of atomic silicon concentration on surfaces of spunbonded web samples of Example 2 versus the number of polyelectrolyte layers deposited thereon.

### DETAILED DESCRIPTION

The present disclosure is directed to fibrous articles having enhanced surface functionality. The fibrous articles of the present disclosure may comprise a nonwoven, woven, or knitted substrate, or a combination thereof such as a stitch-bonded substrate or a laminate of two or more fiber-containing structures and one or more polyelectrolyte layers on fibers of the fibrous substrate so as to provide a desired surface functionality. The present disclosure (global replace) is also directed to methods of making fibrous articles having enhanced surface functionality, as well as methods of using the fibrous articles in a variety of applications including, but not limited to, filtration applications.

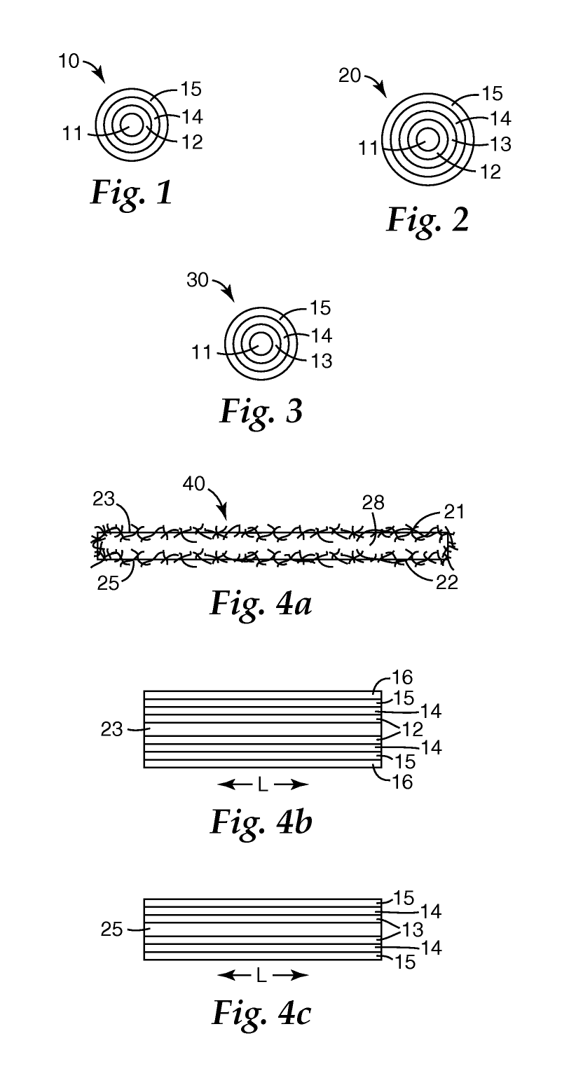

A cross-sectional view of an exemplary fiber within a fibrous article of the present disclosure is shown in FIG. 1. Exemplary fiber 10 of FIG. 1 comprises a fiber 11, a diamond-like film coating layer 12 over fiber 11, and a polyelectrolyte layer 15 bonded to diamond-like film coating layer 12 via a bonding layer 14.

A cross-sectional view of another exemplary fiber within a fibrous article of the present disclosure is shown in FIG. 2. Exemplary fiber 20 of FIG. 2 comprises a fiber 11, a diamond-like film coating layer 12 over fiber 11, an oxygen plasma treatment 13 over diamond-like film coating layer 12, and a polyelectrolyte layer 15 bonded to oxygen plasma treatment 13 via a bonding layer 14.

A cross-sectional view of yet another exemplary fiber within a fibrous article of the present disclosure is shown in FIG. 3. Exemplary fiber 30 of FIG. 3 comprises a fiber 11, an oxygen plasma treatment 13 over fiber 11, and a polyelectrolyte layer 15 bonded to oxygen plasma treatment 13 via a bonding layer 14.

In each of FIGS. 1-3, it should be noted that each of the layers depicted on exemplary fibers 10, 20 and 30 (e.g., diamond-like film coating layer 12, oxygen plasma treatment 13, bonding layer 14, and polyelectrolyte layer 15) may completely surround an outer circumference of a given fiber or may only coat a portion of an outer circumference of a given fiber. Further, it should be noted that each subsequently applied layer or surface treatment depicted on exemplary fibers 10, 20 and 30 (e.g., oxygen plasma treatment 13 over diamond-like film coating layer 12, bonding layer 14 over diamond-like film coating layer 12 or oxygen plasma treatment 13, and polyelectrolyte layer 15 over bonding layer 14) may cover an entire surface area of the previously applied layer or only a portion of the entire surface area of the previously applied layer.

In addition, it should be noted that FIGS. 1-3, and the remaining figures, are not drawn to scale, and layers shown in the figures are utilized to depict various coatings and/or surface treatments having a layer thickness on exemplary fibers 10, 20 and 30, although such coatings and/or surface treatments may have layer thicknesses as small as a few atomic layers in the nanometer scale.

As shown in FIG. 4a, exemplary nonwoven substrate 28 of exemplary nonwoven article 40 has a first major surface 21 and a second major surface 22 opposite first major surface 21. Fibers 23 extend along first major surface 21, while fibers 25 extend along second major surface 22. As discussed below, fibers 23 and fibers 25 may be surface treated so as to have essentially identical surface treatments, different surface treatments, or a combination of a surface treatment and no surface treatment (e.g., fibers 23 are surface treated, but fibers 25 are not surface treated). FIGS. 4b and 4c depict possible surface treatments for fibers 23 and fibers 25 of exemplary nonwoven substrate 28.

As shown in FIG. 4b, along a length L of exemplary fiber 23, the surface treatment comprises diamond-like film coating layer 12 over at least a portion of exemplary fiber 23, polyelectrolyte layer 15 bonded to diamond-like film coating layer 12 via bonding layer 14. In this exemplary embodiment, a second polyelectrolyte layer 16 is shown over polyelectrolyte layer 15. For example, polyelectrolyte layer 15 may comprise an anionic polyelectrolyte layer while second polyelectrolyte layer 16 comprises a cationic polyelectrolyte layer. Further, as shown in FIG. 4c, along a length L of exemplary fiber 25, the surface treatment comprises oxygen plasma treatment 13 over at least a portion of exemplary fiber 25, polyelectrolyte layer 15 bonded to oxygen plasma treatment 13 via bonding layer 14.

The surface treatment options of the present disclosure enable the production of a variety of functionalized nonwoven substrates having desired surface characteristics.

Outermost surfaces of a given fibrous substrate (e.g., major surfaces 21 and 22 of exemplary nonwoven substrate 28) may have similar or different surface characteristics (e.g., surface charge) as described further below.

### I. Fibrous Articles

As shown in FIGS. 1-4c, the fibrous articles of the present disclosure may comprise a number of different components, and layer/surface treatment configurations. A description of possible fibrous article components and fibrous article configurations is provided below.

A. Fibrous Article Components

The fibrous articles of the present disclosure may comprise one or more of the following components.
1. Fibrous Substrate

The fibrous articles of the present disclosure comprise at least one fiber-containing substrate, fabric or web (these terms are used interchangeably to describe the fibrous component). The fibrous substrates comprise natural fibers, synthetic fibers, or combinations thereof. Exemplary natural fibers suitable for forming the fibrous substrate include, but are not limited to, cotton fibers, viscose fibers, wood pulp fibers, cellulose-containing fibers, and combinations thereof. Exemplary synthetic fibers may be formed from any fiber-forming material including, but not limited to, polymeric materials including, but are not limited to, polyolefins such as polypropylene and polyethylene; polyesters such as polyethylene terephthalate and polybutylene terephthalate, polyethylene adipate, polyesters based on polyethylene glycols and dicarboxylic acids such as succinic and adipic acid; polyamide (Nylon-6 and Nylon-6,6); polyurethanes; polybutene; polyhydroxyacid condensation polymers such as polyhydroxyalkanoates such as polylactic acids; polyvinyl alcohol; polyphenylene sulfide; polysulfone; liquid crystalline polymers; polyethylene-co-vinylacetate; polyacrylonitrile; cyclic polyolefins; or any combination thereof. In one exemplary embodiment, the synthetic fibers comprise polypropylene fibers. It also may be useful to treat inorganic fibers in the methods described herein such as fiberglass, aluminum oxide fibers, ceramic fibers, and the like, as well as combinations of inorganic and the above-mentioned organic fibers.

The fibrous substrate may comprise monocomponent fibers comprising any one of the above-mentioned polymers, copolymers or other fiber-forming material. Monocomponent fibers may contain additives as described below, but comprise a single fiber-forming material selected from the above-described fiber-forming materials. The monocomponent fibers typically comprise at least 75 weight percent of any one of the above-described fiber-forming materials with up to 25 weight percent of one or more additives. Desirably, the monocomponent fibers comprise at least 80 weight percent, more desirably at least 85 weight percent, at least 90 weight percent, at least 95 weight percent, and as much as 100 weight percent of any one of the above-described fiber-forming materials, wherein all weights are based on a total weight of the fiber.

The fibrous substrate may also comprise multi-component fibers formed from (1) two or more of the above-described fiber-forming materials and (2) one or more additives as described below. As used herein, the term “multi-component fiber” is used to refer to a fiber formed from two or more fiber-forming materials. Suitable multi-component fiber configurations include, but are not limited to, a sheath-core configuration, a side-by-side configuration, and an “island-in-the-sea” configuration.

For fibrous substrates formed from multi-component fibers, desirably the multi-component fiber comprises (1) from about 75 to about 99 weight percent of two or more of the above-described polymers and (2) from about 25 to about 1 weight percent of one or more additional fiber-forming materials based on the total weight of the fiber.

Each fibrous substrate may have a basis weight, which varies depending upon the particular end use of the article. Typically, each fibrous substrate has a basis weight of less than about 1000 grams per square meter (gsm). In some embodiments, each fibrous substrate has a basis weight of from about 1.0 gsm to about 500 gsm. In other embodiments, each fibrous substrate has a basis weight of from about 10 gsm to about 150 gsm.

As with the basis weight, each fibrous substrate may have a thickness, which varies depending upon the particular end use of the article. Typically, each fibrous substrate has a thickness of less than about 150 millimeters (mm). In some embodiments, each fibrous substrate has a thickness of from about 0.5 mm to about 100 mm. In other embodiments, each fibrous substrate has a thickness of from about 1.0 mm to about 50 mm.

In most embodiments, the fibers within the fibrous substrate are substantially uniformly distributed within the fibrous substrate. However, there may be some embodiments wherein it is desirable to have a non-uniform distribution of fibers within the fibrous substrate.

In addition to the fiber-forming materials mentioned above, various additives may be added to the fiber melt and extruded to incorporate the additive into the fiber. Alternatively, a given additive may be applied onto at least a portion of an outer fiber surface after a fiber extrusion process. Typically, the amount of additives is less than about 25 wt %, desirably, up to about 5.0 wt %, based on a total weight of the fiber. Suitable additives include, but are not limited to, fillers, stabilizers, plasticizers, tackifiers, flow control agents, cure rate retarders, adhesion promoters (for example, silanes and titanates), adjuvants, impact modifiers, expandable microspheres, thermally conductive particles, electrically conductive particles, silica, glass, clay, talc, pigments, colorants, glass beads or bubbles, antioxidants, optical brighteners, antimicrobial agents, surfactants, fire retardants, and fluoropolymers. One or more of the above-described additives may be used to reduce the weight and/or cost of the resulting fiber and layer, adjust viscosity, or modify the thermal properties of the fiber or confer a range of physical properties derived from the physical property activity of the additive including electrical, optical, density-related, liquid barrier or adhesive tack related properties.

The fibrous substrate may be formed using any conventional fabric-forming process. Suitable nonwoven fibrous substrates include, but are not limited to, spunbonded webs, spunlaced webs, meltblown webs, carded webs, needle-punched fabrics, hydroentangled fabrics, unidirectional fiber layer(s), meshes, or combinations thereof. In one desired embodiment, the fibrous substrate comprises a polypropylene nonwoven web, desirably, a polypropylene spunbonded web.

Other fibrous webs which may be useful in the present disclosure include knit and woven fabrics. These fabrics may be formed using yarns based on continuous filaments or made form staple fibers. Typically, knits include warp knits such as raschel and Milanese as well as circular knits and weft knits. Any suitable weft knit may be used such as jersey, rib, double, and purl knits may be used. Pile knits also may be used. Combinations of any of the aforesaid knit structures may be employed.

Typically, woven fabrics are made of two sets of yarns, namely, a lengthwise set which is called the warp and a crosswise set called the filling or weft. In a weaving process, generally the warp yarns are raised and lowered as the weft yarns are inserted creating a grid like structure. Different weaves are possible based on how the weft yarns are inserted relative to the warp yarns such as plain, twill and stain. Warp pile woven fabrics also are suitable. Any suitable woven fabric may be used.
2. Diamond-Like Glass (DLG) Film Coating

The fibrous articles of the present disclosure may further comprise at least one diamond-like glass (DLG) film coating. Each diamond-like glass (DLG) film coating comprises a carbon-rich diamond-like amorphous covalent system containing carbon, silicon, hydrogen and oxygen. Each DLG film coating is created by depositing a dense random covalent system comprising carbon, silicon, hydrogen, and oxygen under ion bombardment conditions by locating a substrate, for example, a fibrous substrate, on a powered electrode in a radio frequency (“RF”) chemical reactor. In one specific embodiment, a DLG film coating is deposited under intense ion bombardment conditions from mixtures of tetramethylsilane and oxygen. Typically, a DLG film coating shows negligible optical absorption in the visible and ultraviolet regions (250 to 800 nm). Also, a DLG film coating usually shows improved resistance to flex-cracking compared to some other types of carbonaceous films and excellent adhesion to many substrates, including ceramics, glass, metals, polymers, and natural fibers.

Each diamond-like glass (DLG) film coating typically contains at least about 30 atomic percent carbon, at least about 25 atomic percent silicon, and less than or equal to about 45 atomic percent oxygen. Each DLG film coating typically contains from about 30 to about 50 atomic percent carbon. In some embodiments, a DLG film coating comprises about 25 to about 35 atomic percent silicon. In other embodiments, the DLG film coating comprises about 20 to about 40 atomic percent oxygen. In some desired embodiments, the DLG film coating comprises from about 30 to about 36 atomic percent carbon, from about 26 to about 32 atomic percent silicon, and from about 35 to about 41 atomic percent oxygen on a hydrogen free basis. As used herein, a “hydrogen free basis” refers to the atomic composition of a material as established by a method such as Electron Spectroscopy for Chemical Analysis (ESCA), which does not detect hydrogen even if large amounts are present in the thin DLG films. (References to compositional percentages herein refer to atomic percents.)

Thin DLG film coatings may have a variety of light transmissive properties. Thus, depending upon the composition, the thin DLG film coatings may have increased transmissive properties at various frequencies. In some embodiments, the thin DLG film coating is at least 50 percent transmissive to radiation at one or more wavelength from about 180 to about 800 nanometers. In other embodiments, the DLG film coating is transmissive to greater than 70 percent (and more desirably greater than 90 percent) of radiation at one or more wavelengths from about 180 to about 800 nanometers.

Each diamond-like-glass film coating typically has a coating thickness on individual fibers of the fibrous substrate of up to about 10 microns (μm). More typically, each diamond-like glass film coating has a coating thickness ranging from about 1 nm to about 10,000 nm, desirably, ranging from about 1 nm to about 100 nm. Each diamond-like glass film coating can be made to a desired specific thickness, typically from 1 to 10 μm, but optionally less than 1 micron or more than 10 microns.

Regardless of the thickness of the DLG film coating, the DLG film coating typically has an extinction coefficient of less than about 0.002 at 250 nm and more typically less than about 0.010 at 250 nm. Also, the DLG film coating usually has a refractive index greater than about 1.4 and sometimes greater than about 1.7. Notably, the DLG film coating shows low levels of fluorescence, typically very low, and sometimes low enough that the DLG film coating shows no fluorescence. Desirably, the DLG film coating has a fluorescence comparable to, nearly equal to, or equal to that of pure quartz.

Diamond-like glass (DLG) film coatings suitable for use in the present disclosure and methods of forming the same are disclosed in U.S. Pat. Nos. 6,696,157, 6,881,538, and 6,878,419, the subject of matter of each of which is incorporated herein by reference in its entirety.

Each diamond-like glass (DLG) film coating may cover fibers extending along an entire major surface of the fibrous substrate, less than an entire major surface of the fibrous substrate, or any portion or all of both major surfaces of the fibrous substrate. In some embodiments, it may be desirable to coat a portion of the fibers extending along a major surface of the fibrous substrate. In these embodiments, a masking layer may be used to provide partial coverage of a major surface of the fibrous substrate. Partial coverage of a major surface of the fibrous substrate may provide a desired pattern, lettering, or any other coating configuration of a diamond-like glass (DLG) film coating on fibers extending along one or both major surfaces of the fibrous substrate.
3. Oxygen Plasma Treatment

The fibrous articles of the present disclosure may further comprise an oxygen plasma treatment on the fibers extending along an entire major surface of the fibrous substrate, less than an entire major surface of the fibrous substrate, or any portion or all of both major surfaces of the fibrous substrate. The oxygen plasma treatment leads to chemical etching of the polymer fibers or DLG film coating and surface modification of the chemical functional groups thereon. A variety of oxygen functional groups such as C—O, C═O, O—C═O, C—O—O and CO₃ are created on the fiber surface or DLG film coating surface as a result of oxygen plasma treatment. A detailed description of the effect of oxygen plasma treatment on polymeric surfaces is provided in C. M. Chan, T. M. Ko, and H. Hiraoko, “Plasma Surface Modification by Plasma and Photons” in Surface Science Reports 24 (1996) 1-54, the subject matter of which is hereby incorporated by reference in its entirety.
4. Polyelectrolyte Layers

The fibrous articles of the present disclosure further comprise at least one polyelectrolyte layer on the above-described diamond-like glass (DLG) film coating or oxygen plasma treatment. As described further below, typically, the fibrous articles comprise alternating polyelectrolyte layers, wherein each polyelectrolyte layer comprises at least one polymeric material having an overall positive or negative charge. As used herein a polyelectrolyte is a polymer having multiple ionizable groups. Generally, the polyelectrolytes have on average at least 3 ionizable groups per molecule and preferably have greater than 10 ionizable groups per molecule and most preferably at least 20 ionizable groups per molecule on average. This may be determined from the polymer composition and the weight average molecular weight. Such polyelectrolytes may comprise permanently charged groups such as quaternary amines or alternatively may be comprised of polymers having multiple acidic or basic groups or a combination thereof. The polyelectrolytes comprised of multiple acidic groups may comprise carboxylate, phosphate, phosphonate sulfate, sulfonate groups, as well as combinations thereof. The polyelectrolytes comprised of basic groups may comprise primary, secondary, and tertiary amines, as well as combinations thereof, and also optionally in combination with quaternary amine groups.

Furthermore, certain polyelectrolytes may comprise both anionic (acidic) or cationic (basic or quaternary amine) groups and would therefore be zwitterionic. Examples of basic groups include, but are not limited to, primary, secondary, or tertiary amines, which, upon neutralization, form protonated amino groups. Examples of acidic groups, which, upon neutralization, form anionic groups, include, but are not limited to, hydrogen sulfate (—OSO₂OH), sulfonic acid (—SO₂OH), hydrogen phosphate ((—O)₂P(O)OH or —OP(O)(OH)₂ or —OP(O)(OH)O⁻M⁺), phosphonic acid (—PO(OH)₂ or —PO(OH)O⁻M⁺), and carboxylic acid (—CO₂H). In these formulae, M is a positively charged counterion and is selected from the group consisting of hydrogen, sodium, potassium, lithium, ammonium, calcium, magnesium or N⁺R′₄ where each R′ is independently an alkyl group of 1 to 4 carbon atoms optionally substituted with N, O, or S atoms.

The resulting fibrous article may possess an outer surface having an overall positive charge, an overall negative charge, or both at a pH where optimum complexation occurs between the layers. It is recognized that polyacid and polybasic polymers will have a pH dependent charge density. For polyacid and polybasic polyelectrolytes the polymer will be charged when at least a portion of the acid or basic groups are neutralized. The pH at which this occurs is dependent on the pKa of the acid or basic groups. Generally, speaking it is preferred that at least 10% of the groups be neutralized, preferably at least 50% and most preferably at least 90%. This can be easily determined from the pH of the in use environment and the pKa of the acidic or basic groups on the polyelectrolyte. Exemplary synthetic and natural materials for forming a given polyelectrolyte layer having an overall positive charge include, but are not limited to, cationic polyelectrolytes including poly(allylamine) (PAH), polydiallyldimethyl ammonium halide (such as the chloride salt PDDAC), linear and branched poly(ethylenimime), polyaminoamides, quaternary ammonium natural polymer derivatives such as quaternized derivatives of cellulose, guar, and other gums and polybasic polysaccharides such as chitosan, net basic proteins such as gelatin, pectin and the like. Many other suitable quaternary ammonium polymers are suitable for use in the present disclosure and include those known as “polyquaternium” polymers in references such as the Cosmetic Bench Reference, the subject matter of which is hereby incorporated by reference.

The molecular weight of the synthetic polyelectrolyte molecules is typically in the range of about 1,000 to about 5,000,000 grams/mole, more desirably from about 5,000 to about 1,000,000 grams/mole. For naturally-occurring polyelectrolyte molecules, molecular weights can be as high as 10,000,000 grams/mole.

Exemplary synthetic and natural materials for forming a given polyelectrolyte layer having an overall negative charge include, but are not limited to, homopolymers or copolymers of acrylic acid, methacrylic acid, maleic anhydride, itaconic acid, citraconic acid and the like, with acrylic acid being one preferred monomer. The polymeric resin can also comprise other comonomers that are polymerizable with a carboxylic acid-containing monomer, such as methyl vinyl ether, lower alkyl(meth)acrylates, and the like. Exemplary polymers include, but are not limited to, poly(styrene sulfonic acid) (PSS), poly(vinylsulfonic acid), polyacrylic acid (PAA), polymethacrylic acid (PMA), poly(2-acrylamido-2-methylpropane sulfonic acid), and poly(anetholesulfonic acid). Natural and modified natural anionic polymers are also suitable for use in the present disclosure and include carboxylic acid containing polysaccharides such as hyaluronic acid, chondroitin sulfate, dextran sulfate, carboxymethylcellulose, carboxymethyl chitosan, carboxymethyl starch, carboxymethyl dextran, alginic acid, heparin, DNA, RNA, and the like. Various salts of these polymers may be employed including salts of mono- or polyvalent metals such as alkali earth metals, calcium, magnesium, aluminum and the like.

The molecular weight of the synthetic polyelectrolyte molecules are typically in the range of 1,000 to about 5,000,000 grams/mole, but preferably about 5,000 to about 1,000,000 grams/mole. For the naturally occurring polyelectrolyte molecules their molecular weights could be as high as 10,000,000 grams/mole.

It also may be desirable to incorporate other nonionic polymers or small molecules in one or more polyelectrolyte layers to help control the subsequent dissolution rate. Thus, for example, natural or synthetic nonionic polymers added to the polyelectrolyte solution(s) include, but are not limited to, polymers such as polyethylene oxide, polyethylene glycol, polyvinyl alcohol, water-soluble polyacrylates such as poly(hydroxyethyl acrylates), methylcellulose, dextran, glycerol, hydroxypropyldextran, hydroxypropylcellulose, hydroxypropyl starch, polypropylene glycol, ethylhydroxy-ethylcellulose, polyvinylpyrolidone, modified gaur and other gums, and the like.

A given polyelectrolyte layer may be applied in the form of an aqueous solution typically comprising up to about 10 wt % of one or more polyanions or polycations and typically about 90 wt % or greater of water. Typically, the aqueous solution comprises from about 0.01 to about 10.0 wt % of one or more polyanions or polycations and from about 99.99 to about 90 wt % of water. In other embodiments, the aqueous solution comprises from about 0.01 to about 1.0 wt % of one or more polyanions or polycations, and from 99.99 to about 99.0 wt % of water.

Like the above-described diamond-like glass (DLG) film coating or oxygen plasma treatment, a given polyelectrolyte layer may cover an entire surface of the diamond-like glass (DLG) film coating (or oxygen plasma treatment) or less than an entire surface of the diamond-like glass (DLG) film coating (or oxygen plasma treatment). In some embodiments, it may be desirable to cover only a portion of a diamond-like glass (DLG) film coating (or oxygen plasma treatment). In these embodiments, a masking layer may be used to provide partial coverage of a diamond-like glass (DLG) film coating (or oxygen plasma treatment). Partial coverage of a diamond-like glass (DLG) film coating (or oxygen plasma treatment) with a polyelectrolyte layer may provide a desired pattern, lettering, or any other coating configuration on the diamond-like glass (DLG) film coating (or oxygen plasma treatment).
5. Active Ingredients

The fibrous articles of the present disclosure may further comprise one or more active ingredients incorporated into the above-described polyelectrolyte layer(s). Active ingredients may include, but are not limited to, antimicrobial materials such as silver-containing compounds, copper-containing compounds, and iodine-containing compounds. When present, one or more active ingredients may be incorporated into a given polyelectrolyte layer by forming an aqueous polyelectrolyte solution as described above and blending therein at least one active ingredient. The resulting aqueous solution typically comprises from about 0.01 to about 10.0 wt % of one or more polyanions or polycations, from about 99.99 to about 90 wt % of water, and from about 0.001 to about 2.0 wt % of one or more active ingredients. Alternatively, an aqueous solution containing one or more active ingredients may be applied to a fibrous article after the formation of one or more of the above-described polyelectrolyte layer(s). In this embodiment, the resulting aqueous solution typically comprises from about 99.999 to about 98 wt % of water, and from about 0.001 to about 2.0 wt % of one or more active ingredients.
6. Bonding Layers

The fibrous articles of the present disclosure may comprise one or more bonding layers so as to enhance bonding of a given polyelectrolyte layer to a diamond-like glass (DLG) film coating or oxygen plasma treatment. Suitable bonding layers comprise any bonding composition capable of bonding to (i) a diamond-like glass (DLG) film coating or oxygen plasma treatment and (ii) a polyelectrolyte layer.

In one exemplary embodiment, the bonding layer comprises a silane coupling agent. Suitable silane coupling agents include, but are not limited to, silanes containing amino groups, mercapto groups, or hydroxyl groups. Exemplary aminosilanes include, but are not limited to, 3-aminopropyltrimethoxysilane, 3-aminopropyltriethoxysilane; N-(2-aminoethyl)-3-aminopropyltrimethoxysilane; 3-aminopropylmethyldiethoxysilane; 3-aminopropyltris(2-(2-methoxyethoxy)ethoxy)silane; 3-aminopropyltriisopropenyloxy-silane; 3-aminopropyltri(butanone oximo)silane; 4-aminobutyltriethoxysilane; N²-(aminoethyl)-3-aminopropyltris(2-ethylhexoxy)silane; 3-aminopropyldimethylethoxy-silane; 3-aminopropyldiisopropylethoxysilane; and 3-aminopropylphenyldiethoxysilane. Exemplary mercaptosilanes include, but are not limited to, 3-mercaptopropyltrimethoxysilane (MPTS). Exemplary hydroxysilanes include, but are not limited to, bis(2-hydroxyethyl)-3-aminopropyl-triethoxysilane. In one desired embodiment, the bonding layer comprises 3-aminopropyltriethoxysilane or 3-amino-propyltrimethoxysilane.

When used as a bonding layer, the silane coupling agent may be applied as a bonding layer in the form of an aqueous solution typically comprising up to about 10 wt % of one or more silane coupling agents and typically about 90 wt % or greater of water or alcohol. Typically, the aqueous solution comprising from about 0.5 to about 10.0 wt % of one or more silane coupling agents and from about 99.5 to about 90 wt % of water or alcohol.

Like the above-described diamond-like glass (DLG) film coating (or oxygen plasma treatment), the bonding layer may cover an entire surface of the diamond-like glass (DLG) film coating (or oxygen plasma treatment) or less than an entire surface of the diamond-like glass (DLG) film coating (or oxygen plasma treatment). In some embodiments, it may be desirable to cover only a portion of a diamond-like glass (DLG) film coating (or oxygen plasma treatment). In these embodiments, a masking layer may be used to provide partial coverage of a diamond-like glass (DLG) film coating (or oxygen plasma treatment). Partial coverage of a diamond-like glass (DLG) film coating (or oxygen plasma treatment) may provide a desired pattern, lettering, or any other coating configuration on the diamond-like glass (DLG) film coating (or oxygen plasma treatment).

In one exemplary embodiment, the fibrous article of the present disclosure comprises a fibrous substrate (e.g., a nonwoven substrate) comprising fibers along first and second major surfaces of the fibrous substrate; a first diamond-like glass film coating at least a portion of the fibers along the first major surface, the second major surface, or both; a first silane coupling layer on the first diamond-like glass film; and an anionic polyelectrolyte layer on the first silane coupling layer. Additional layers such as one or more cationic polyelectrolyte layers and additional anionic polyelectrolyte layers may be provided on the fibrous substrate. For example, in one exemplary embodiment, the fibrous article further comprises at least one cationic polyelectrolyte layer on the at least one anionic polyelectrolyte layer. In one desired embodiment, the anionic polyelectrolyte layer comprises poly(styrene sulfonic acid) sodium salt, and the cationic polyelectrolyte layer comprises poly(allylamine hydrochloride).

In a further embodiment, the silane coupling layer comprises an aminosilane coupling agent, and the silane coupling layer is subjected to an acidic solution so as to protonate amino groups on the silane coupling layer. The protonated amino groups enhance bonding to a subsequently applied polyanion layer, such as a layer containing poly(styrene sulfonic acid) sodium salt.
7. Additional Optional Layers

The fibrous articles of the present disclosure may further comprise one or more additional layers in combination with the above-described fibrous substrate, one or more diamond-like glass (DLG) film coatings, one or more oxygen plasma treatments, one or more bonding layers, and one or more polyelectrolyte layers. One or more additional layers may be present over at least a portion of an outer surface of the fibrous substrate, an outer surface of a diamond-like glass (DLG) film coating, an outer surface of an oxygen plasma treatment, an outer surface of a bonding layer, an outer surface of a polyelectrolyte layer, or any combination thereof.

Suitable additional layers include, but are not limited to, a color-containing layer (e.g., a print layer), (color can optionally be added to one of the polyelectrolyte layers as well); an adhesive layer (e.g., a pressure-sensitive adhesive (PSA) layer, a heat activatable adhesive layer, or a combination thereof); foams; gels, layers of particles; foil layers; films; other fiber-containing layers (e.g., woven, knitted, or nonwoven layers); membranes (i.e., films with controlled permeability, such as dialysis membranes, reverse osmosis membranes, etc.); netting; mesh; or a combination thereof

B. Fibrous Article Configurations

In its simplest form, the fibrous articles of the present disclosure comprise a fibrous substrate comprising fibers along first and second major surfaces of the fibrous substrate; a diamond-like glass film coating and/or an oxygen plasma treatment on at least a portion of the fibers along the first major surface; and an anionic polyelectrolyte layer bonded to the first diamond-like glass film coating or oxygen plasma treatment. However, as shown in FIGS. 4a-c, the fibrous articles may comprise a variety of surface treatments and/or additional layers resulting in many possible article configurations. A description of some exemplary article configurations is provided below.
1. DLG Film Coating and/or Oxygen Plasma Treatment On One Major Surface

In some embodiments of the present disclosure, the fibrous articles comprise a surface treatment on one major surface of a fibrous substrate, wherein the surface treatment comprises a first diamond-like glass film coating and/or an oxygen plasma treatment, a bonding layer, and one or more polyelectrolyte layers. In one exemplary embodiment, the first polyelectrolyte layer bonded to the first diamond-like glass film coating or oxygen plasma treatment comprises an anionic polyelectrolyte layer. Desirably, the first anionic polyelectrolyte layer is bonded to the first diamond-like glass film coating or oxygen plasma treatment via a first silane coupling layer on the first diamond-like glass film coating or oxygen plasma treatment. The first anionic polyelectrolyte layer provides an overall negative surface charge to the resulting fibrous article.

The fibrous article may further comprise a first cationic polyelectrolyte layer on the first anionic polyelectrolyte layer so as to provide an overall positive surface charge to the resulting fibrous article. In some embodiments, it may be desirable to provide numerous alternating anionic and cationic polyelectrolyte layers on the fibrous article to build a multi-layered construction on the major surface of the fibrous article.

Although any of the above-mentioned polycations and polyanions may be used to form alternating anionic and cationic polyelectrolyte layers, in one desired embodiment, the fibrous article comprise alternating anionic polyelectrolyte layers comprising poly(styrene sulfonic acid) sodium salt, and cationic polyelectrolyte layers comprising poly(allylamine hydrochloride). The resulting fibrous article may have an outermost cationic polyelectrolyte layer or an outermost anionic polyelectrolyte layer.

In embodiments wherein the fibrous substrate comprises a diamond-like glass film coating and/or oxygen plasma treatment on only one major surface, the other major surface may be uncoated or may comprise any number of additional layers such as those described above.
2. DLG Film Coatings and/or Oxygen Plasma Treatments On Both Major Surfaces

In other embodiments of the present disclosure, the fibrous articles comprise a diamond-like glass film coating and/or oxygen plasma treatment on both major surfaces of the fibrous substrate. In addition to a first diamond-like glass film coating and/or first oxygen plasma treatment, a first bonding layer, and one or more polyelectrolyte layers on the first bonding layer, the fibrous article further comprises a second diamond-like glass film and/or second oxygen plasma treatment on at least a portion of the fibers along a second major surface of the fibrous substrate; a second bonding layer (e.g., a silane layer) deposited on the second diamond-like glass film or second oxygen plasma treatment; and one or more polyelectrolyte layers on the second bonding layer. The one or more polyelectrolyte layers on the second bonding layer may comprise an anionic polyelectrolyte layer, or both an anionic polyelectrolyte layer and a cationic polyelectrolyte layer.

The first surface treatment on a first major surface of the fibrous substrate may be similar to or different from the second surface treatment on a second major surface of the fibrous substrate. In one exemplary embodiment, the fibers along the first major surface of the fibrous substrate are coated with a first chemistry of layers and the fibers along the second major surface are coated with a second chemistry, wherein the first chemistry differs from the second chemistry.

### II. Method of Making Fibrous Articles

The present disclosure is further directed to methods of making fibrous articles having enhanced surface functionality. In one exemplary embodiment, the method of making a fibrous article comprises subjecting a fibrous substrate having first and second major surfaces to a surface treatment process so as to provide a fiber surface treatment over at least a portion of fibers along the first major surface, wherein the fiber surface treatment comprises (i) an oxygen plasma treatment, (ii) a diamond-like glass film coating, or both (i) and (ii); and bonding at least one polyelectrolyte layer to the fiber surface treatment. The method of making a fibrous article may further comprise one or more additional steps so as to provide fibrous articles having a desired surface chemistry. A description of possible method steps is provided below.

A. Fibrous Substrate Formation

As discussed above, fibrous substrates suitable for use in the present disclosure may be formed using any conventional fabric-forming process. Suitable process steps include any process step used in the formation of conventional nonwoven, knit and woven fabrics. Suitable process steps for forming a nonwoven substrate include, but are not limited to, process steps for forming spunbonded webs, spunlaced webs, meltblown webs, carded webs, needle-punched fabrics, hydroentangled fabrics, unidirectional fiber layer(s), meshes, or combinations thereof. Suitable process steps for forming a knit substrate include, but are not limited to, process steps for forming a warp knit, weft knit, or any other conventional knit fabric. Suitable process steps for forming a woven substrate include, but are not limited to, process steps for weaving.

Further, it should be noted that any of the above-mentioned fibers for forming a given fibrous substrate may be treated with one or more of the above-described coating materials prior to being formed into a fibrous substrate. For example, fibers may be subjected to a DLG coating treatment, an oxygen plasma treatment, a silane treatment, a polyelectrolyte treatment, or any combination thereof, prior to being formed into a nonwoven, knit or woven substrate using any of the above-mentioned conventional fabric-forming steps (e.g., a carding step, a stitch-bonding step, a knitting step, and a weaving step).

B. Plasma Deposition of DLG Film Coating

As discussed above, diamond-like glass (DLG) film coatings suitable for use in the present disclosure and methods of forming the same are disclosed in U.S. Pat. Nos. 6,696,157, 6,881,538, and 6,878,419, the subject of matter of each of which is incorporated herein by reference in its entirety.

This method step typically comprises providing a capacitively coupled reactor system having two electrodes in an evacuable reaction chamber. The chamber is partially evacuated, and radio frequency power is applied to one of the electrodes. A carbon and silicon containing source is introduced between the electrodes to form a plasma including reactive species in proximity to the electrodes, and to also form an ion sheath proximate at least one electrode. The fibrous substrate is placed within the ion sheath or passes adjacent to the electrode, and is exposed to the reactive species to form a diamond-like glass on the fibrous substrate. The conditions can result in a thin film on the fibers of the fibrous substrate that comprises, for example, a diamond-like structure having on a hydrogen-free basis at least 30 atomic percent carbon, at least 25 atomic percent silicon, and less than 45 atomic percent oxygen. The thin film can be made to a specific thickness by adjusting dwell time within the chamber or by administering multiple deposition steps.

Species within the plasma react on the fibrous substrate surface (e.g., fiber surface) to form covalent bonds, resulting in the amorphous diamond-like glass film on the surface of the fibrous substrate. Multiple fibrous substrates may be simultaneously coated with DLG during a given process step. The fibrous substrates can be held in a vessel or container within an evacuable chamber that is capable of maintaining conditions that produce diamond-like film deposition. Alternatively, the fibrous substrate can be passed through the vacuum chamber. That is, the chamber provides an environment which allows for the control of, among other things, pressure, the flow of various inert and reactive gases, voltage supplied to the powered electrode, strength of the electric field across the ion sheath, formation of a plasma containing reactive species, intensity of ion bombardment, and rate of deposition of a diamond-like glass film from the reactive species.

Prior to the deposition step, the chamber is evacuated to the extent necessary to remove air and any impurities. Inert gases (e.g., argon) may be admitted into the chamber to alter pressure. Once the fibrous substrate(s) is placed in the chamber and the chamber evacuated, a substance containing carbon and silicon, desirably including a carbon-containing gas, and optionally a substance from which an additional component or components can be deposited, is admitted into the chamber and, upon application of an electric field, forms a plasma from which the diamond-like glass film is deposited. At the pressures and temperatures of diamond-like film deposition (typically 0.13 to 133 Pa (0.001 to 1.0 Torr) (all pressures stated herein are gauge pressure) and less than 50° C.), the carbon and silicon-containing substances and substances from which an optional additional component may be obtained will be in their vapor form.

If hydrogen is to be included in the diamond-like glass film, hydrocarbons are particularly desired as a source for the carbon and hydrogen. Suitable hydrocarbons include, but are not limited to, acetylene, methane, butadiene, benzene, methylcyclopentadiene, pentadiene, styrene, naphthalene, azulene, and mixtures thereof. Sources of silicon include, but are not limited to, silanes such as SiH₄, Si₂H₆, tetramethylsilane, and hexamethyldisiloxane. Gases containing optional additional components can also be introduced to the reaction chamber. Gases with low ionization potentials, i.e., 10 electronVolts (eV) or less, typically are used for efficient deposition of the DLG film coating. The additional optional diamond-like glass film components, including one or more of hydrogen, nitrogen, oxygen, fluorine, sulfur, titanium, or copper, are introduced in vapor form into the reaction chamber during the deposition process. Typically, even when the sources for the additional components are solids or fluids, the reduced pressure in the chamber will cause the source to volatilize. Alternatively, the additional components may be entrained in an inert gas stream. The additional components may be added to the chamber while a carbon or hydrocarbon-containing gas is sustaining the plasma and/or may be added to the chamber after the flow of carbon or hydrocarbon-containing gas has been stopped.

Sources of hydrogen include hydrocarbon gases and molecular hydrogen (H₂). Sources of fluorine include compounds such as carbon tetrafluoride (CF₄, sulfur hexafluoride (SF₆), perfluorobutane (C₄F₁₀), C₂F₆, C₃F₈, and C₄F₁₀. Sources of oxygen include oxygen gas (O₂), hydrogen peroxide (H₂O₂), water (H₂O), and ozone (O₃). Sources of nitrogen include nitrogen gas (N₂), ammonia (NH₃), and hydrazine (N₂H₆). Sources of sulfur include sulfur hexafluoride (SF₆), sulfur dioxide (SO₂), and hydrogen sulfide (H₂₅). Sources of copper include copper acetylacetonate. Sources of titanium include titanium halides such as titanium tetrachloride.

An ion sheath is necessary to obtain ion bombardment, which, in turn, is necessary to produce a densely packed diamond-like film. An explanation of the formation of ion sheaths can be found in Brian Chapman, Glow Discharge Processes, 153 (John Wiley & Sons, New York 1980), the subject of which is hereby incorporated by reference in its entirety.

The electrodes may be the same size or different sizes. If the electrodes are different sizes, the smaller electrode will have a larger ion sheath (regardless of whether it is the grounded or powered electrode). This type of configuration is referred to as an “asymmetric” parallel plate reactor. An asymmetric configuration produces a higher voltage potential across the ion sheath surrounding the smaller electrode. Establishing a large ion sheath on one of the electrodes is desired because the fibrous substrate is desirably located within an ion sheath to benefit from the ion bombardment effects that occur within the sheath.

Desired electrode surface area ratios are from about 2:1 to about 4:1, and more desirably from about 3:1 to about 4:1. The ion sheath on the smaller electrode will increase as the ratio increases, but beyond a ratio of about 4:1 little additional benefit is achieved. The reaction chamber itself can act as an electrode. A desired configuration includes a powered electrode within a grounded reaction chamber that has two to three times the surface area of the powered electrode.

In an RF-generated plasma, energy is coupled into the plasma through electrons. The plasma acts as the charge carrier between the electrodes. The plasma can fill the entire reaction chamber and is typically visible as a colored cloud. The ion sheath appears as a darker area around one or both electrodes. In a parallel plate reactor using RF energy, the applied frequency should be in the range of 0.001 to 100 MHz, desirably about 13.56 MHz or any whole number multiple thereof. This RF power creates a plasma from the gas (or gases) within the chamber. The RF power source can be an RF generator such as a 13.56 MHz oscillator connected to the powered electrode via a network that acts to match the impedance of the power supply with that of the transmission line and plasma load (which is usually about 50 ohms so as to effectively couple the RF power). Hence this is referred to as a matching network.

The ion sheath around the electrodes causes negative self-biasing of the electrodes relative to the plasma. In an asymmetric configuration, the negative self-bias voltage is negligible on the larger electrode and the negative bias on the smaller electrode is typically in the range of about 100 to about 2000 volts. While the acceptable frequency range from the RF power source may be high enough to form a large negative direct current (DC) self bias on the smaller electrode, it should not be high enough to create standing waves in the resulting plasma, which is inefficient for the deposition of a DLG film.

For planar fibrous substrates, deposition of dense diamond-like glass thin films is normally achieved in a parallel plate reactor by placing the fibrous substrate in direct contact with a powered electrode, which is made smaller than the grounded electrode. This allows the fibrous substrate to act as an electrode due to capacitive coupling between the powered electrode and the fibrous substrate. This is described in M. M. David, et al., Plasma Deposition and Etching of Diamond-Like Carbon Films, AIChE Journal, vol. 37, No. 3, p. 367 (1991), the subject matter of which is incorporated herein by reference. In the case of an elongate fibrous substrate, the fibrous substrate is optionally continuously pulled through the chamber, passing proximate the electrode with the largest ion sheath, while a continuous RF field is placed on the electrode and sufficient carbon-containing gas is present within the chamber. A vacuum is maintained at the inlet and exit of the chamber by two roughing pumps. The result is a continuous DLG film coating on an elongated fibrous substrate, and substantially only on the fibrous substrate.

In one exemplary embodiment, the fibrous substrate (e.g., nonwoven substrate) to be coated has first and second major surfaces, and the DLG film deposition step provides a diamond-like-glass film on the first major surface, the second major surface, or both first and second major surfaces of the fibrous substrate. In one desired embodiment, the deposition step comprises depositing a silicon-containing diamond-like film via a plasma deposition process onto the fibers of a fibrous substrate; and subsequently treating the silicon-containing diamond-like film in an oxygen plasma treatment to form silanol groups onto a surface of the diamond-like film. See, for example, the examples below.

C. Oxygen Plasma Treatment of a DLG Film Coating or Fiber Surface

As discussed above, a diamond-like glass (DLG) film coating or a fiber surface may be subjected to an oxygen plasma treatment. The oxygen plasma treatment is performed in a similar manner as described above for the plasma deposition of a DLG film coating except that only an oxygen source, such as those described above, is used to treat a diamond-like glass (DLG) film coating surface or a fiber surface. See, for example, the exemplary oxygen plasma treatment described in the examples below.

D. Bonding of Polyelectrolyte Layer(s)

The methods of forming a fibrous article further comprise bonding at least one polyelectrolyte layer to the diamond-like glass film coating or the oxygen plasma treatment. Typically, the step of bonding a polyelectrolyte layer to the diamond-like glass film coating or the oxygen plasma treatment comprises applying a bonding layer to the diamond-like glass film coating or the oxygen plasma treatment, and overcoating the bonding layer with a polyelectrolyte layer. In one exemplary embodiment, the bonding step comprises coupling a silane coupling agent to the diamond-like-glass film or the oxygen plasma treatment; and overcoating the silane coupling agent with at least one polyelectrolyte layer.

The step of applying a bonding layer to the diamond-like glass film coating or the oxygen plasma treatment typically comprises applying an aqueous solution comprising one more bonding agents to the diamond-like glass film coating or the oxygen plasma treatment. For example, an aqueous solution comprising one or more silane coupling agents may be formed and then coated onto the diamond-like glass film coating or the oxygen plasma treatment using any conventional coating method. Suitable coating methods include, but are not limited to, dip coating, spray coating, die-coating etc.

When the bonding agent comprises an aminosilane, the bonding step may further comprise protonating amino groups of the silane coupling agent by treating the silane coupling agent with (i) a polyelectrolyte solution having a negative charge and a reduced pH or (ii) an aqueous solution having a reduced pH. The pH range may be from 0 to about 5, more desirably, from 0 to about 3. The aminosilane may be protonated by applying an acidic solution having an acid molar concentration of up to about 0.02 M, and comprising any acid including, but not limited to, hydrochloric acid.

Once a desired bonding layer is applied to the diamond-like-glass film or the oxygen plasma treatment, a polyelectrolyte layer may be applied over the bonding layer. In one exemplary embodiment, the bonding layer has positively charged moieties along an outer surface of the bonding layer, and the first polyelectrolyte layer applied thereon comprises an anionic polyelectrolyte layer, such as an anionic polyelectrolyte layer comprising at least one of the above-described polyanions. In other exemplary embodiments, the bonding layer may have negatively charged moieties along an outer surface of the bonding layer, and the first polyelectrolyte layer applied thereon comprises a cationic polyelectrolyte layer, such as a cationic polyelectrolyte layer comprising at least one of the above-described polycations.

The step of applying a polyelectrolyte layer onto a bonding layer typically comprises applying an aqueous solution comprising one more polyanions or polycations with one or more optional active ingredients to the bonding layer. For example, an aqueous solution comprising one or more polyanions and one or more optional active ingredients may be formed and then coated onto the bonding layer using any conventional coating method such those described above with reference to coating of the bonding layer.

In one exemplary embodiment, at least one polyelectrolyte layer is applied onto the bonding layer (e.g., a protonated aminosilane layer), wherein the at least one polyelectrolyte layer comprises (i) an anionic polyelectrolyte layer, or (ii) an anionic polyelectrolyte layer and a subsequently applied cationic polyelectrolyte layer. In a further exemplary embodiment, at least one polyelectrolyte layer is applied onto the bonding layer, wherein the at least one polyelectrolyte layer comprises alternating anionic and cationic polyelectrolyte layers so as to provide an outermost anionic polyelectrolyte layer, an outermost cationic polyelectrolyte layer, or both along first and second major surfaces of the fibrous substrate with any or all of the polyelectrolyte layers containing one or more optional active ingredients as described above.

E. Bonding of Additional Layer(s)

The methods of forming a fibrous article may further comprise bonding at one or more additional layers to the fibrous substrate or a layer thereon. Any conventional method may be used to bond an additional layer to the fibrous substrate or a layer thereon including, but not limited to, a coating step such as those disclosed above, a lamination step, an extrusion step, etc. It should be noted that one or more additional layers may be bonded to the fibrous substrate prior to or after the above-described DLG film deposition step, the oxygen plasma treatment step, and/or the polyelectrolyte application step.

### III. Method of Using Fibrous Articles

The fibrous articles may be used in a variety of applications including, but not limited to, filtration, microbial detection, wound healing products, drug delivery, bioprocessing (e.g., protein purification), permselective materials for protective coatings, food safety, anti-glare and anti-fog materials for medical use, etc.

### EXAMPLES

This disclosure may be illustrated by way of the following examples.

### Materials:

Spunbonded polypropylene nonwoven web—prepared by The 3M Company using polypropylene commercially available under the trade designation FINA 3860 PP from FINA, Inc. (Houston, Tex.);

100% carded cotton nonwoven web commercially available under the trade designation WEBRIL® wipes, style 142-951, basis wt—258 gsm, from BBA Nonwovens (Green Bay, Wis.).

Poly(styrene sulfonic acid) sodium salt (PSS)—a polyanion solution having a molecular weight (MW) of 70,000 commercially available from Alfa Aesar (Ward Hill, Mass.);

Poly(allylamine hydrochloride) (PAH)—a polycation solution having a molecular weight (MW) of 60,000 commercially available from Alfa Aesar (Ward Hill, Mass.);

(3-aminopropyl)trimethoxysilane purchased from Gelest, Inc. (Morrisville, Pa.) and used as received—an aminosilane used was a 2 wt % solution of (3-aminopropyl)trimethoxysilane in 98 wt % water;

Polyhexamethylene biguanide hydrochloride (PHMB)—a polycation solution having antimicrobial properties commercially available from ICI Americas (Bridgewater, N.J.) under the trade designation COSMOCIL® CQ and used as received;

Povidone Iodine—a polycation when in solution having antimicrobial and antiseptic properties commercially available from International Specialty Products, (Wayne, N.J.), and used as received.

Sodium Alginate—a polyanion when in solution commercially available as MANUCOL™ LF from International Specialty Products, (Wayne, N.J.), and used as received.

Purified water—water that was purified using a Millipore Direct Q system with a resistivity of 18.2 MΩ-cm;

Tetramethylsilane—tetramethylsilane, 99.9% NMR grade, commercially available from Sigma-Aldrich Chemicals (St. Louis, Mo.); and

Oxygen—oxygen, 99.99% UHP Grade, commercially available from Scott Specialty Gases (Plumsteadville, Pa.).

### Test Methods:

X-ray Photoelectron Spectroscopic (XPS) Technique

X-ray photoelectron spectroscopic (XPS) techniques were used to analyze the surface characteristics of coated spunbonded web samples. X-ray photoelectron spectroscopic techniques enabled interrogation of a sample's surface characteristics within a depth of 2.5 nm so as to obtain an atomic concentration of a coating material on the sample surface. The spectra were taken and recorded at a 15° take-off angle between a plane of the sample surface and the entrance lens of the optical detector using an XPS apparatus commercially available from Kratos Analytical, (Kratos Axis Ultra) of Manchester, England, UK.

Microbial Testing Protocol

The disc assay used to test the material was based on two agar diffusion methods; Bauer-Kerby and Minimal Inhibitory Concentration, MIC, standard number M-100. These two methods are found in the National Committee for Clinical Laboratory Standards, NCCLS.

The agar used for the disc assay was EasyGel™ Media, item #3093-55—Total Count purchased from Webster Scientific (Hamilton, N.J.). The bacteria used for the inoculation of the EasyGel™ Media were MRSA (ATCC #33593) and E. coli (ATCC #53500) purchased from American Type Culture Collection (ATCC) (Manassas, Va.). Discs were formed by punching out a 12.5 mm disc, in duplicate, using a die cutter with a diameter width of 12.5 mm.

The two bacteria cultures used for the EasyGel™ Media inoculation were grown separately in Tryptic Soy Broth, TSB, @ 35° C. for 18-24 hours. The cultures were taken from maintenance cultures stored in the cooler at 4° C. After cultures were grown at 35° C. for 24 hours, the concentration of bacteria produced was approximately 10⁸. These cultures were serially diluted to concentrations of 10⁶ bacteria. The bacterial concentrations were then diluted one more time as the bacteria was added to bottles producing 1:10 dilution or a concentration of 10⁵ in the gel bottles. The bacterial cultures were not mixed and were added to the bottles separately. The bottles containing the inoculated gel were poured into specially coated Petri dishes and allowed to set for 45 minutes. This produced two sets of agar plates: one with MRSA and the other set containing E. coli.

Once the agar was set, the treated and control discs were placed on the two inoculated surface in the center on each Petri dish. Two sets of inoculated agars were used to determine if the treated disc worked better on gram positive, MRSA, or gram negative bacteria, E. coli. The plates were than placed in an incubator for 18-24 hours at 35° C. to allow bacteria growth. After 18-24 hours, the plates were read using a Craftsman Digital Caliper. If there was no diffusion of the antimicrobial from the disc, i.e., no clear area around the disc was formed, the result was called a “no zone.” If there was diffusion from the disc, i.e., a clear zone area was formed, the diameter of the zone area was measured and recorded. The amount of release from a given treated disc determined the resulting zone diameter.

Zone of Inhibition Test Protocol

The zone of inhibition assay (ZOI Assay) was used to evaluate the anti-microbial activity of the web substrates. Testing was performed by preparing separate solutions of Staphylococcus aureus (ATCC 6538) and Pseudomonas aeruginosa (ATCC 9027) at a concentration of approximately 1×10⁸ colony forming units (cfu) per milliliter (ml) in Phosphate Buffered Saline (PBS) using a 0.5 McFarland Equivalence Turbidity Standard. The solution was used to prepare a bacterial lawn by dipping a sterile cotton applicator into the solution and swabbing the dry surface of a trypticase soy agar (TSA) plate in three different directions. Samples were prepared by cutting a 7-millimeter disk of the test material. Three disks from each sample of the test material were then placed with the active side down onto an inoculated plate of each organism and pressed firmly against the agar with sterile forceps to ensure complete contact with the agar. The plates were incubated at 28° C.±1° C. for 24 hours. The area under and surrounding the samples were examined for bacterial growth. The ZOI Assay provides both a qualitative (amount of growth beneath sample) and quantitative (size of zone in mm) measure, with the magnitude of the zone being a measure of the intrinsic antibacterial efficacy of the material.

### Example 1

A spunbonded polypropylene nonwoven web was prepared using FINA 3860 polypropylene. The spunbonded web had the following properties: a basis weight of 117 gsm, a thickness of 1.27 mm (50 mils), and an effective fiber diameter of 13.7 μm. The polypropylene nonwoven sample sheets were first treated in a plasma reactor (PLASMA-THERM™ Model 3082 available from Plasma-Therm, Inc. (Kresson, N.J.).

The plasma reactor was used to deposit a silicon-containing layer onto spunbonded web samples by using a gas mixture of tetramethylsilane (99.9% NMR grade) and oxygen (99.99% UHP Grade). The chamber was pumped by a roots blower (Edwards, Model EH1200 (Sussex, England)) backed by a dry mechanical pump (Edwards, Model iQDP80 (Sussex, England)). Plasma was powered by a 13.56 MHz radio frequency power supply (RF Plasma Products, Model RF50S (city, state)).

The nonwoven samples were located on the powered electrode to deposit a diamond-like film by ion bombardment. The chamber was pumped down to a base pressure of 10 mTorr before the plasma deposition step was started. A two-step deposition procedure was employed. First, a silicon-containing diamond-like film was deposited, and then silicon-containing diamond-like film was treated in an oxygen plasma to impart silanol groups onto the surface of the silicon-containing diamond-like film. The process conditions used in these two steps were as follows:

### Step 1: Silicon-Containing Diamond-Like Film Deposition

| | | |
|---|---|---|
| Tetramethylsilane Flow Rate | 150 | sccm |
| Oxygen Flow Rate | 500 | sccm |
| Process Pressure | 50 | mTorr |
| Plasma Power | 2000 | watts |
| Treatment Time | 12 | seconds |

### Step 2: Post Oxygen Plasma Treatment

| | |
|---|---|
| Oxygen Flow Rate | 500 sccm |
| Pressure | 150 mTorr |
| Plasma Power | 300 watts |
| Treatment Time | 60 seconds |

After completing steps 1 and 2 above, the chamber was vented and the samples were flipped around to treat a back side of each sample in the same two-step procedure described above.

The plasma-treated non-woven samples rich in silanol groups were then treated in a 2 wt % (3-aminopropyl)trimethoxysilane (ATS)/98 wt % solution by dipping each sample in the solution for a few minutes. The treated samples were dried over-night in an oven at about 60° C.

For all samples, the first polyelectrolyte layers were deposited on the DLG/aminosilane treated samples by dipping each sample in an acidified 0.02 M (based on molecular weight of polymer repeat unit) solution of PSS having a pH of 1.50 for about 1 hour. The acidified PSS solution protonated the amino groups in the silane coating layer enabling electrostatic interaction between the polyanion layer and the cationic surface of the treated sample.

Excess PSS solution was removed by rinsing each sample with two aliquots of ultra-pure water for about 1-2 minutes each. Then, each sample was dipped into an acidic 0.02 M (based on molecular weight of polymer repeat unit) solution of PAH having a pH of 2.00 for 30 minutes. Excess PAH solution was removed by rinsing each sample with two aliquots of ultra-pure water for about 1-2 minutes each to complete a first bi-layer (i.e., polyanion layer in combination with polycation layer).

For the deposition of subsequent layers, each sample was dipped in solutions of PSS (having a pH of 5.56) or PAH (having a pH of 2.00) for 30 minutes each. This process continued with consecutive layer-by-layer deposition until ten layers (i.e., five bilayers) were deposited onto the spunbonded web samples.

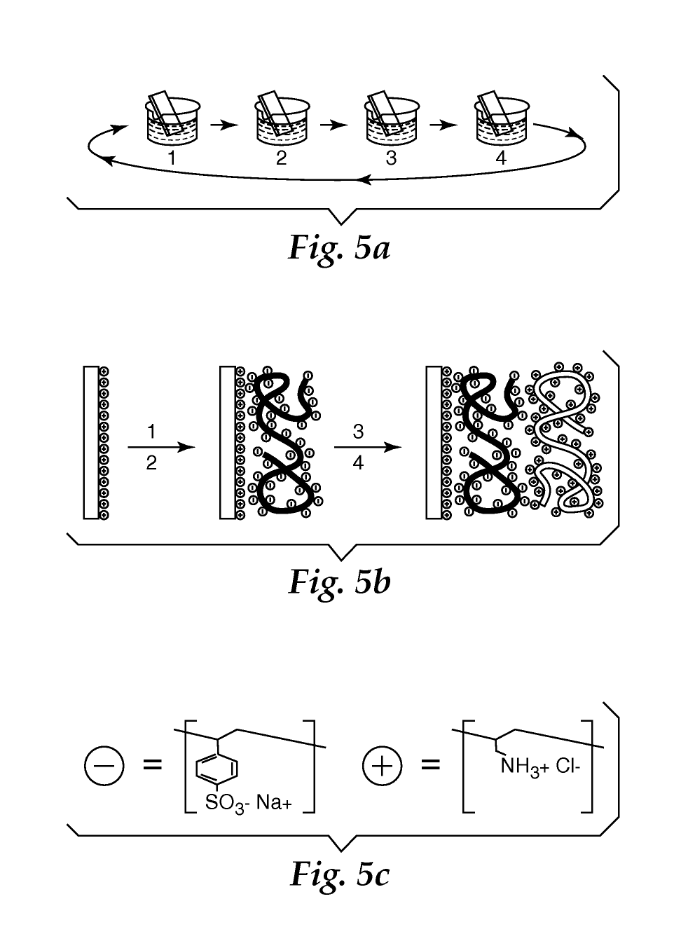

Consequently, spunbonded web samples having an outermost PSS layer had an odd number of layers, while spunbonded web samples having an outermost PAH layer had an even number of layers on at least one side of the sample. FIGS. 5a-c provide a schematic illustration of the above-described layer-by-layer deposition process. As shown in FIG. 5a-b, solution 1 provided a polyanion layer onto the positively charged surface of the nonwoven substrate, the positively charged surface being the result of the above-described ATS treatment step. Solution 2 provided a wash to remove residual polyanion layer components. Solution 3 provided a polycation layer on the negatively charged surface of the polyanion layer. Solution 4 provided a wash to remove residual polycation layer components. As shown in FIG. 5c, the polyanion solution provided negative charges resulting from dissociation of the sodium cation from poly(styrene sulfonic acid) sodium salt, while the polycation solution provided positive charges resulting from dissociation of the chloride anion from poly(allylamine hydrochloride).

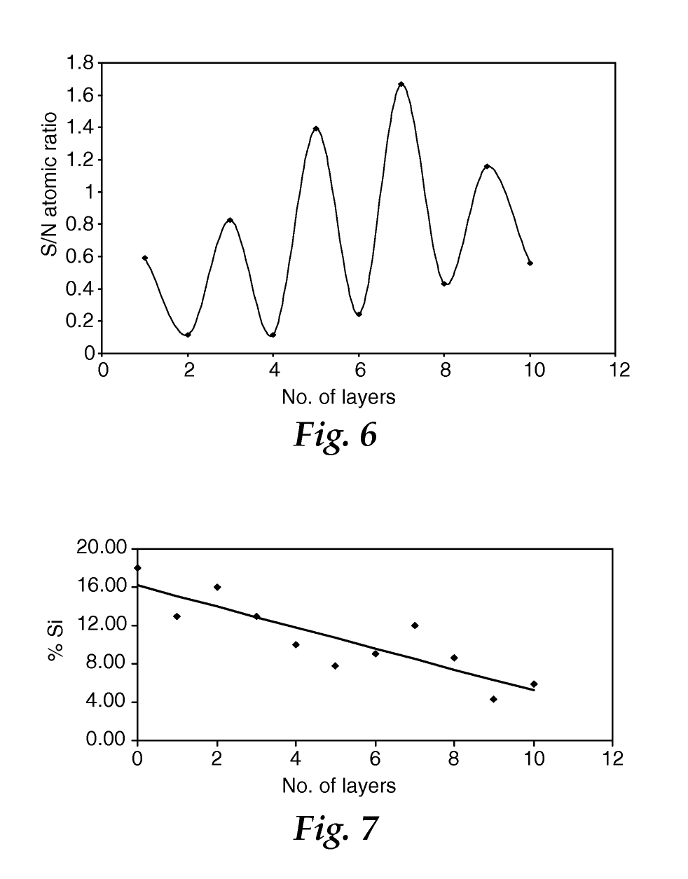

Surface characteristics of the resulting coated substrates were measured using the above-described XPS techniques. The results are presented in FIGS. 6 and 7.

FIG. 6 provides a plot of the sulfur:nitrogen atomic ratio of a polyelectrolyte layer against the number of deposition layers. As shown in FIG. 6, a pronounced odd-even trend was observed. The S:N ratio is comparatively higher when the outer-layer is PSS, and lower when the outer-layer is PAH. The plot in FIG. 6 indicates that the layers are stratified even though the layers may not be tightly packed mono-layers. Further evidence of the fabrication of a polyelectrolyte multi-layer structure is provided in FIG. 7.

Given that the priming of each sample substrate attaches an amino-silane group onto the surface, the relative amount of detectable atomic silicon as the number of layers of polyelectrolyte are added provides evidence that a polyelectrolyte multi-layer structure is produced using the above-described deposition technique. As shown in FIG. 7, as the number of polyelectrolyte layers increases, the amount of detectable atomic silicon decreases on the surface of the spunbonded web samples.

From the results shown in FIGS. 6-7, confirmation of multiple polyelectrolyte layers on the fibers of the polypropylene spunbonded substrate is confirmed.

### Example 2

A polyelectrolyte structure bonded to a polypropylene spunbonded nonwoven web via an oxygen plasma and an aminosilane treatment was prepared as follows.

A spunbonded polypropylene nonwoven web as used in Example 1 was treated in a plasma reactor as described in Example 1 to surface treat fibers of the spunbonded polypropylene nonwoven web with an oxygen plasma treatment. Nonwoven samples were treated in an oxygen plasma to generate polar oxygen groups such as C—O, C═O, O—C═O, C—O—O and CO₃ on the surface of the fibers. The process conditions used in the oxygen plasma treatment step were as follows:

| | |
|---|---|
| Oxygen Flow Rate | 500 sccm |
| Pressure | 150 mTorr |
| Plasma Power | 300 watts |
| Treatment Time | 60 seconds |

After completing the oxygen plasma treatment step, the chamber was vented and the samples were flipped around to treat a back side of each sample using the same procedure and process conditions described above.

The plasma-treated non-woven samples rich in hydroxyl groups/polar oxygen groups were then treated in a 2 wt % (3-aminopropyl)trimethoxysilane (ATS)/98 wt % solution by dipping each sample in the solution for a few minutes. The treated samples were dried over-night in an oven at about 60° C. Fourier Transform Infrared Spectroscopy (FTIR) was used to verify the presence of aminosilane on fiber surfaces of each sample after washing.

For all samples, the first polyelectrolyte layers were deposited on the oxygen plasma/aminosilane treated samples by dipping each sample in an acidified 0.02 M solution of PSS having a pH of 1.50 for about 1 hour. The acidified PSS solution protonated the amino groups in the silane coating layer enabling electrostatic interaction between the polyanion layer and the cationic surface of the treated sample.

Excess PSS solution was removed by rinsing each sample with two aliquots of ultra-pure water for about 1-2 minutes each. Then, each sample was dipped into an acidic 0.02 M solution of PAH having a pH of 2.00 for 30 minutes. Excess PAH solution was removed by rinsing each sample with two aliquots of ultra-pure water for about 1-2 minutes each to complete a first bi-layer (i.e., polyanion layer in combination with polycation layer).

For the deposition of subsequent layers, each sample was dipped in 0.02 M solutions of PSS (having a pH of 5.34) or PAH (having a pH of 2.00) for 30 minutes each. This process continued with consecutive layer-by-layer deposition until ten layers (i.e., five bilayers) were deposited onto the spunbonded web samples. Each sample was then rinsed with two aliquots of ultra-pure water for about 1-2 minutes each and dried at room temperature under vacuum.

Spunbonded web samples having an outermost PSS layer had an odd number of layers, while spunbonded web samples having an outermost PAH layer had an even number of layers on at least one side of a given sample.

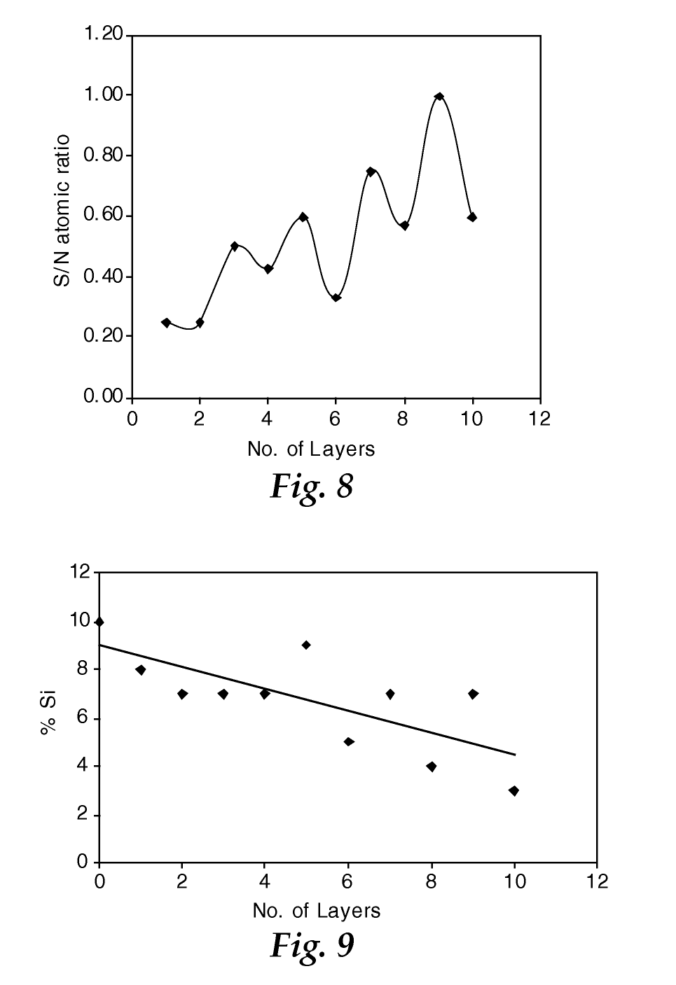

Surface characteristics of the resulting coated substrates were measured using the above-described XPS techniques. The results are presented in FIGS. 8 and 9. FIG. 8 provides a plot of the sulfur:nitrogen atomic ratio of a given polyelectrolyte layer against the number of deposition layers. As shown in FIG. 8, a pronounced odd-even trend was observed. The S:N ratio is comparatively higher when the outer-layer is PSS, and lower when the outer-layer is PAH. The plot in FIG. 8 indicates that the layers are stratified even though the layers may not be tightly packed mono-layers. Further evidence of the fabrication of a polyelectrolyte multi-layer structure is provided in FIG. 9.

Given that the priming of each sample substrate attaches an aminosilane group onto the fiber surface, the relative amount of detectable atomic silicon as the number of layers of polyelectrolyte are added provides evidence that a polyelectrolyte multi-layer structure is produced using the above-described deposition technique. As shown in FIG. 7, as the number of polyelectrolyte layers increases, the amount of detectable atomic silicon decreases on the surface of the spunbonded web samples.

From the results shown in FIGS. 8-9, confirmation of multiple polyelectrolyte layers on the fibers of the polypropylene spunbonded substrate is confirmed.

### Example 3

A poly(hexamethylene biguanide)/poly(styrene sulfonate) polyelectrolyte multilayer (PEM) structure on a polypropylene spunbonded substrate suitable for use as a wound dressing material was prepared using a layer-by-layer technique as described below.

A surface treated spunbonded polypropylene nonwoven web was prepared as described in Example 1 so as to form DLG/aminosilane treated samples. Each sample was then coated with polyelectrolyte layers comprising poly(styrene sulfonate) and poly(hexamethylene biguanide) layers.

For all samples, the first polyelectrolyte layers were deposited on the DLG/aminosilane treated samples by dipping each sample in an acidified 0.02 M solution of PSS having a pH of about 2.0 for about 1 hour. The acidified PSS solution protonated the amino groups in the silane coating layer enabling electrostatic interaction between the polyanion layer and the cationic surface of the treated sample. Excess PSS solution was removed by rinsing each sample with two aliquots of ultra-pure water for about 1-2 minutes each.

A 20% w/v solution of poly(hexamethylene biguanide) (PHMB) was used as received. The 20% PHMB solution was then diluted to form 1%, 3% or 5% w/v solutions of PHMB. Initial pH values of each solution were lowered to a pH of about 2.00.

Each sample was dipped into the 1%, 3% or 5% w/v solutions of PHMB for about 20 minutes. Excess PHMB solution was removed by rinsing each sample with two aliquots of ultra-pure water for about 1-2 minutes each to complete a first bi-layer (i.e., polyanion layer in combination with polycation layer).

For the deposition of any subsequent layers, each sample was dipped in 0.02 M solutions of PSS (having a pH of 1.55) or PHMB (having a pH of 2.00) for 20 minutes each. This process continued with consecutive layer-by-layer deposition until up to 8 layers (i.e., four bilayers) were deposited onto the spunbonded web samples. Each sample was then rinsed with two aliquots of ultra-pure water for about 1-2 minutes each and dried at a temperature of no more than 40° C. under vacuum. All spunbonded web samples had an outermost PHMB layer.

Using the above-described Microbial Testing Protocol, the antimicrobial properties of the spunbonded web samples were determined. As shown in Table 1 below, the spunbonded web samples provided

TABLE 1
Antimicrobial Properties of Surface Treated Spunbonded Web Samples

| Sample | Mean (mm) | Composition |
|---|---|---|
| PSS/PHMB-1-1 | 13.4 | 1% PHMB (1 bilayer) |
| PSS/PHMB-1-2 | 15.75 | 1% PHMB (2 bilayers) |
| PSS/PHMB-1-3 | 13.1 | 1% PHMB (3 bilayers) |
| PSS/PHMB-1-4 | 24.3 | 1% PHMB (4 bilayers) |
| PSS/PHMB-3-1 | 12.75 | 3% PHMB (1 bilayer) |
| PSS/PHMB-3-2 | 22.6 | 3% PHMB (2 bilayers) |
| PSS/PHMB-3-3 | 27.2 | 3% PHMB (3 bilayers) |
| PSS/PHMB-3-4 | 13.4 | 3% PHMB (4 bilayers) |
| PSS/PHMB-5-1 | 14.935 | 5% PHMB (1 bilayer) |
| PSS/PHMB-5-2 | 23.7 | 5% PHMB (2 bilayers) |
| PSS/PHMB-5-3 | 14.20 | 5% PHMB (3 bilayers) |
| Control - Plain | 12.5 (no change) | No PHMB |
| PP web | | |

### Example 4

Nonwoven 100% carded cotton web samples from BBA (Style WEBRIL™ 142-951), basis wt: 258 gsm, were first treated in a plasma reactor (PLASMA-THERM™ Model 3082 available from Plasma-Therm, Inc. (Kresson, N.J.) using the process parameters as described above in Example 1. Front and back sides of each sample were treated using the same two-step procedure as described above in Example 1.

The plasma-treated non-woven samples rich in silanol groups were then treated in a 2 wt % (3-aminopropyl)trimethoxysilane (ATS)/98 wt % solution by dipping each sample in the solution for a few minutes. The treated samples were dried over-night in an oven at about 60° C.

For all samples, polyelectrolyte layers were deposited on the DLG/aminosilane treated samples as described in Example 1 so as to form alternating PSS and PAH layers until ten layers (i.e., five bilayers) were deposited onto the 100% carded cotton web samples.

Consequently, carded web samples having an outermost PSS layer had an odd number of layers, while carded web samples having an outermost PAH layer had an even number of layers on at least one side of the sample.

Surface characteristics of the resulting coated substrates were measured using the above-described XPS techniques. A plot of the sulfur:nitrogen atomic ratio of a polyelectrolyte layer against the number of deposition layers similar to the plot shown in FIG. 6 was observed. Further, a plot of the amount of detectable atomic silicon versus the number of polyelectrolyte layers also indicated a decrease of detectable atomic silicon on the surface of the carded web samples similar to the plot shown in FIG. 7. Testing confirmed the presence of multiple polyelectrolyte layers on the fibers of the carded web samples.

### Example 5

A silver ion-containing polyelectrolyte multi-layer (PEM) structure on a polypropylene spunbonded substrate suitable for use as a wound dressing material was prepared using a layer-by-layer technique as described below.

A surface treated spunbonded polypropylene nonwoven web was prepared as described in Example 1 so as to form DLG/aminosilane treated samples. Each sample was then coated with a polyelectrolyte layer comprising poly(styrene sulfonate) by dipping each sample in an acidified 0.02 M solution of PSS having a pH of about 2.0 for about 1 hour. Excess PSS solution was removed by rinsing each sample with two aliquots of ultra-pure water for about 1-2 minutes each. The samples were then soaked in a silver solution for one hour and then removed and dried in a vacuum oven. The substrates prepared in this way were termed “wet” samples, while the substrates that were dried following PSS deposition, but before soaking in silver solution were termed “dry” samples.

The silver solution was prepared by dissolving 0.5 grams silver (I) oxide (Ag₂O) in 50 grams of a 10 wt % ammonium carbonate aqueous solution. Results of the zone of inhibition assay testing are shown in Tables 2 and 3 below. An average of three disks per sample was used.

TABLE 2
(Staphylococcus aureus - ATCC 6538)

| | | | Activity under |
|---|---|---|---|
| Sample | 1° Zone (mm) | 2° Zone (mm) | sample |
| Example 5 - wet | none | none | light |
| Example 5 - dry | none | 9.7 | no growth |

TABLE 3
(Pseudomonas aeruginosa - ATCC 9027)

| | | | Activity under |
|---|---|---|---|
| Sample | 1° Zone (mm) | 2° Zone (mm) | sample |
| Example 5 - wet | 7.7 | none | no growth |
| Example 5 - dry | 9.0 | none | no growth |

### Example 6

A silver ion-containing polyelectrolyte multi-layer (PEM) structure on a polypropylene spunbonded substrate suitable for use as a wound dressing material was prepared using a layer-by-layer technique as described in Example 5 except a silver (II) solution was used. The silver solution was prepared by dissolving 0.5 grams silver (II) oxide (AgO) in 50 grams of a 10 wt % ammonium carbonate aqueous solution. Results of the zone of inhibition assay testing are shown in Tables 4 and 5 below. An average of three disks per sample was used.

TABLE 4
(Staphylococcus aureus - ATCC 6538)

| | | | Activity under |
|---|---|---|---|
| Sample | 1° Zone (mm) | 2° Zone (mm) | sample |
| Example 6 - wet | 8.7 | 12.0 | no growth |
| Example 6 - dry | 8.7 | 12.3 | no growth |

TABLE 5
(Pseudomonas aeruginosa - ATCC 9027)

| | | | Activity under |
|---|---|---|---|
| Sample | 1° Zone (mm) | 2° Zone (mm) | sample |
| Example 6 - wet | 10.0 | none | no growth |
| Example 6 - dry | 9.7 | none | no growth |

### Examples 7-10

An iodine-containing polyelectrolyte multi-layer (PEM) structure on a polypropylene spunbonded substrate suitable for use as a wound dressing material was prepared using a layer-by-layer technique as described below.

A surface treated spunbonded polypropylene nonwoven web was prepared as described in Example 1 so as to form DLG/aminosilane treated samples. Each sample was then coated with a sodium alginate layer by dipping each sample in a 0.5 wt % solution of sodium alginate having a pH of 5.24 for about 20 minutes. Excess sodium alginate solution was removed by rinsing each sample with two aliquots of ultra-pure water for about 1-2 minutes each. The samples were then soaked in a 6 wt % povidone iodine (PVP-I) solution having a pH of 2.00 for 20 minutes and then removed and rinsed. The consecutive layer-by-layer deposition process was continued resulting in a series of samples having 1, 2, 3, and 4 sets of bi-layers of sodium alginate and PVP-I. The multi-layer coated samples were dried in a vacuum oven at 40° C. The samples were evaluated for their antimicrobial properties using the Microbial Testing Protocol. The results are shown in Table 6 below.

TABLE 6

| | Zone diameter | Zone diameter |
|---|---|---|
| Example | (mm) MRSA | (mm) E. coli |
| 7 (1 bilayer) | 21.2 | 15.3 |
| 8 (2 bilayers) | 23.4 | 16.5 |
| 9 (3 bilayers) | 24.5 | 16.0 |
| 10 (4 bilayers) | 21.6 | 23.3 |

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of the appended claims and any equivalents thereto.

1. An article comprising:
a fibrous substrate comprising fibers along first and second major surfaces of said fibrous substrate;
a fiber surface treatment over at least a portion of the fibers along said first major surface, said fiber surface treatment comprising (i) an oxygen plasma treatment, or (ii) a diamond-like glass film coating, or both (i) and (ii); and
an anionic polyelectrolyte layer bonded to said fiber surface treatment.

2. The article of claim 1, wherein said fiber surface treatment comprises a diamond-like glass film.

3. The article of claim 2, wherein said first diamond-like glass film comprises a dense random covalent system comprising, on a hydrogen-free basis, at least about 30 atomic percent carbon, at least about 25 atomic percent silicon, and less than or equal to about 45 atomic percent oxygen.

4. (canceled)

5. The article of claim 1, wherein said fiber surface treatment comprises said oxygen plasma treatment.

6. The article of claim 1, wherein said anionic polyelectrolyte layer is bonded to said fiber surface treatment via a silane coupling layer on said fiber surface treatment.

7. The article of claim 1, further comprising:
a cationic polyelectrolyte layer on said anionic polyelectrolyte layer.

8. The article of claim 1, wherein the anionic polyelectrolyte layer comprises poly(styrene sulfonic acid) sodium salt.

9. The article of claim 7, wherein the cationic polyelectrolyte layer comprises poly(allylamine hydrochloride) or poly(hexamethylene biguanide) (PHMB).

10. The article of claim 1, wherein said article comprises alternating anionic polyelectrolyte layers and cationic polyelectrolyte layers.

11. The article of claim 1, wherein said article comprises an outermost cationic polyelectrolyte layer.

12. The article of claim 1, further comprising:
a second fiber surface treatment over at least a portion of the fibers along said second major surface, said second fiber surface treatment comprising (i) an oxygen plasma treatment, or (ii) a diamond-like glass film coating, or both (i) and (ii);
a second silane layer deposited on said second fiber surface treatment; and
an anionic polyelectrolyte layer, or both an anionic polyelectrolyte layer and a cationic polyelectrolyte layer on said second silane layer.

13. The article of claim 12, wherein said fibers along said first major surface are coated with a first chemistry of layers and said fibers along said second major surface are coated with a second chemistry, wherein the first chemistry differs from the second chemistry.

14. (canceled)

15. The article of claim 1, further comprising at least one active ingredient comprising a silver-containing compound, a copper-containing compound, an iodine-containing compound, or a combination thereof.

16. A wound dressing comprising the article of claim 15.

17. An article comprising:
a fibrous substrate comprising fibers along first and second major surfaces of said fibrous substrate;
a diamond-like glass film coating at least a portion of the fibers along said first major surface, said second major surface, or both;
a silane coupling layer on said first diamond-like glass film; and
an anionic polyelectrolyte layer on said silane coupling layer.

18. The article of claim 17, further comprising at least one cationic polyelectrolyte layer on said at least one anionic polyelectrolyte layer.

19. The article of claim 17, wherein said silane coupling layer is subjected to an acidic solution so as to protonate amino groups on said silane coupling layer.

20. (canceled)

21. A method of making a functionalized fibrous substrate, said method comprising:
subjecting a fibrous substrate having first and second major surfaces to a surface treatment process so as to provide a fiber surface treatment over at least a portion of fibers along said first major surface, said fiber surface treatment comprising (i) an oxygen plasma treatment, or (ii) a diamond-like glass film coating, or both (i) and (ii); and
bonding at least one polyelectrolyte layer to the fiber surface treatment.

22. The method of claim 21, wherein said bonding step comprises:
coupling a silane coupling agent to the fiber surface treatment; and
overcoating the silane coupling agent with at least one polyelectrolyte layer, and
optionally, protonating amino groups of the silane coupling agent by treating the silane coupling agent with an acid.

23. (canceled)

24. (canceled)

25. (canceled)

26. (canceled)

27. The method of claim 21, further comprising:
incorporating at least one active ingredient in the at least one polyelectrolyte layer, the at least one active ingredient comprising a silver-containing compound, a copper-containing compound, an iodine-containing compound, or a combination thereof.
